(19) United States
(12) Patent Application Publication
(10) Pub. No.: US 2017/0312116 A1
(43) Pub. Date: Nov. 2, 2017

(54) A METHOD AND DEVICE FOR EXTERNAL URINARY INCONTINENCE TREATMENT FOR WOMEN

(71) Applicant: GR Dome Medical Ltd., Tirat Carmel (IL)

(72) Inventor: Amir Laniado, Haifa (IL)

(21) Appl. No.: 15/522,297

(22) PCT Filed: Nov. 5, 2014
(86) PCT No.: PCT/IL2014/000057

§ 371 Date: Apr. 27, 2017

(51) Int. Cl. A61F 5/455; A61F 5/443

(52) U.S. Cl. CPC A61F 5/4553; A61F 5/443; A61F 5/4556

(57) ABSTRACT

The invention is an external urinary incontinence treatment method and device for women that assists women or a care giver in locating the orifice of the urethral tract for the connection of a tube for removal of urine. The method and device facilitate a stable and reversibly fixated connection of a urine receiving component with the skin surrounding the orifice of the urethral tract of a treated patient while minimizing the discomfort of the treatment. The device for the treatment in women of urinary incontinence comprises a urine collecting component and a stabilizing component that are reversibly connected in a manner that the distance between the components can easily be altered so as to minimize the discomfort caused to a treated patient.

### FIELD OF THE INVENTION

The present invention relates to a method and device for external urinary incontinence treatment for women. More specifically, the present invention relates to a method and device for an external urinary incontinence treatment for women by precisely positioning and stabilizing a urine receiving component around the skin surrounding the external urethral orifice of a treated patient while minimizing the discomfort of the treatment.

### BACKGROUND OF THE INVENTION

The preferred treatment of urinary incontinence in women it is often the use of a device that externally connects to the body of the treated patient and collects urine directly from the external orifice of the uretheral tract without penetrating into the orifice. Typically in the various external incontinence treatment devices that have been described in publications, a component that directly receives the urine from the urethral orifice without penetrating the orifice is utilized. The component directs the urine without leaks to a tube that drains the urine to a collection vessel, typically a disposable bag. The urine receiving component in the text that follows is also referred to interchangeably as the “receiving component”.

In women, the urethra tract is short compared to males and located at the bottom of the pelvis. The urethra tract is about 3.5-5 cm long and exits the body between the clitoris and the vagina. The women external urethral orifice is located 1-2 cm below the clitoris behind the symphysis pubis. The entire length of the urethra tract is embedded in the anterior vaginal wall and it is slightly curved with the concavity directed downwards. Because of the anatomic boundaries many patients and care givers find it difficult to locate the orifice of the urethral tract. The “hidden” location and the moist and cramped surrounding of the lips of the labia minora make the placing and the stabilization of a urine collecting component over the orifice of the urethral tract a difficult task.

In connecting an external urinary incontinence treatment device in women the convenience of the patient is to be considered. A treatment device should have a spatial configuration of the receiving component as well as the spatial configuration of the body-connection components that will minimize the discomfort of the treated patient yet provide a urine leak-tight connection with the urethral tract of the patient. The term “discomfort” is defined by the Merriam-Webster dictionary (http://www.merriam-webster.com/dictionary/pressure) as: “an uncomfortable or painful feeling in the body”. The term “leak-tight connection” refers to a connection in which no urine leaks to the surrounding from the location of the connection between the treatment device and the skin of the treated patient in the course of the urinary incontinence treatment. The tightness in which the receiving component is reversibly fastened to the body of the treated patient as well as the manner in which the receiving component is stabilized when in contact with the patient play a substantial role in determining the discomfort caused to the patient.

The ability of external urinary incontinence treatment devices to collect urine while causing the patient minimal discomfort relates in addition to the spatial configuration of the urine receiving component also to the manner in which the receiving component is reversibly connected, adjusted, fastened and fixated in the genital region, near and/or over the orifice of the urethra. The term “adjusted” in context of the receiving component of the present invention refers to minor placement movements of the component to comfortably-connect to the skin in the near of the orifice of the urethra. The skin in the near of the orifice of the urethra refers to the skin that surrounds the vicinity of the orifice. The term “fastened” in context of the receiving component of the present invention, is used to describe the tightening of the receiving component to the skin surface in the near of the orifice of the urethra of a treated patient in manner that produces a reversible urine leak-free connection. The term “genital region” refers to skin area in the near and including the labia majora of the treated patient. The term “pressure” is defined by the Merriam-Webster dictionary (http://www.merriam-webster.com/dictionary/pressure) as “a force that is produced when something presses or pushes against something else”.

An example of reversibly connecting a receiving component of an external urinary incontinence treatment device to genital region of women is described in UK patent application GB2,015,347 (inventors: Steer et al.). Steer et al. describes a receiving component in the form of a pad of surgical adhesive material, which is connected to a funnel and a pipe. The pipe may include a non-return valve to prevent backflow of urine. The pad may have a ridge-like projection centrally thereon constructed to extend between the labia majora of the wearer. The surgical adhesive material connects and fixates the device to the body of the patient. No reference is made to easily and regularly adjusting, fastening and fixating the external urinary incontinence treatment device during prolonged usage periods so as to cause minimal discomfort to a treated patient

Another example of connecting and fastening of a urine receiving component in an external urinary incontinence treatment device to the genital region of women is given in U.S. Pat. No. 5,263,947 (Kay) which describes a device which comprises a receiving-component (referred to as: “housing” in Kay's text) having an outlet conduit, a plurality of leaves made of a thin, vapor permeable film connected to the housing, a vapor permeable adhesive layer on the interior surface of the leaves, a microbial-barrier layer provided on an inner surface of the housing and a barrier disc provided on the inner surface of the housing. The reversible connection and fixation to genital region of women is done by the receiving-component around the urethral orifice and applying the adhesive layer to the skin surface in a wrinkle-free manner. In addition, retaining straps are connected to the housing of the device and are worn and fastened to the lower torso of the patient to stabilize the housing position. The use of adhesive layer connections with no additional ability of regularly adjusting, fastening and fixating of the housing to the surrounding of the urethral orifice without reconnecting the adhesive layer, will cause the treated patient inconvenience when in use for extended time periods. In addition, the straps are cumbersome to ware and adjust, especially in a lying down position.

Another example of the connecting and fastening of a urine receiving component in an external urinary incontinence treatment device of women is given in PCT/IL2014/000039 (Laniado). Laniado describes a device that comprises: a urine receiving component, a tube, a receiving component supporting element, a tube locking system, a genital region connection component, and a genital region anchoring element. The tube connects and communicates freely with the urine receiving component and is inserted through the receiving component supporting element. The tube also communicates with said tube locking system and is able to move vertically the receiving component supporting element when said tube locking system is deactivated. The tube is fixated in its movement at a desired position along the length of said tube when said tube locking system is activated. The device receiving component supporting element is connected to the genital region connection component and with the tube locking system. The genital region connection component is connected to the genital region anchoring element. In deployment of the device, the genital region anchoring element is reversibly connected to the skin of the genital region of the treated patient, and urine receiving-component is connected to the skin surrounding the urethral orifice of the treated patient when the tube is moved towards the genital region of the patient. The tube is reversibly fixated in place by the tube locking system after urine receiving component is adjusted and fastened to the skin surrounding the urethral orifice of the patient in a urine leak free connection while applying the minimal required pressure, thus, causing the treated patient minimal inconvenience. The tube lucking system can be easily deactivated and reactivated to adjust the connection of the receiving component to the skin surrounding the urethral orifice in accordance to changing body postures of the patient. While PCT/IL2014/000039 relates to the ability to adjust and fasten the urine receiving component to the skin of the treated patient so as to cause minimal discomfort, there is no mention of assisting the patient or care giver in finding the orifice of the urethral tract and positioning the receiving component in the proper location. In addition, in PCT/IL2014/000039 the manner in which the device is connected to the body of the treated patient the receiving component is stabilized in place around the orifice of the urethral orifice in a reversible connection that requires substantial tightness that could cause discomfort. An external urinary incontinence treatment method and device for women of the present invention, facilitate the easy locating of the external orifice urethral tract of a treated patient. In addition, the method and device of the present invention facilitate the reversible connecting of a receiving component to the skin that surrounds the urethral orifice in a manner that is east to deploy yet the connection is stable and easy to regularly adjust, fasten and fixate in accordance with the desire of the treated patient or care giver.

### SUMMARY OF THE INVENTION

The external urinary incontinence treatment method and device for women of the present invention assists women or a care giver in locating the orifice of the urethral tract. In addition, the method and device, facilitate a stable and reversibly fixated connection of a urine receiving component with the skin surrounding the orifice of the urethral tract of a treated patient

The device for the treatment in women of urinary incontinence comprises: a urine collecting component, and a stabilizing component.

The urine collecting component is produced of rigid or semi rigid material or materials such as but not limited to, silicone, plastic or rubber.
The stabilizing component is produced of rigid or semi rigid material or materials such as, but not limited to, silicone, plastic or rubber and also includes sheets of material or materials produced from synthetic and/or natural material origin such as cotton and polyester materials.

Each of the components (individually) can be produced as to have a single entity (produced with all the components already connected) or they can be made of various components that are produced separately and are assembled together to form the components.

The urine collecting component comprises (designated below as a to c):

a) A urine receiving component that has a dome shaped structure, typically, but not limited to, in an elongated configuration. The dome of the urine receiving component has a spatial configuration in which the rim of the dome facilitates a connection to the skin surrounding the orifice of the urethral tract of a treated patient in a urine leak-tight connection. The dome structure has a tube entry hole at one of its sides and optionally, an air-inlet hole that is typically located at its other side. The air-inlet hole runs through the wall of the dome structure. Optionally, the air-inlet hole can be in the tube (instead of in urine receiving component dome) or in other locations in the urine receiving component.

b) A tube that connects to the hole in the dome structure and communicates freely with the volume enclosed in the urine receiving component. A tube-entry-port connects to the tip of the tube not connected to the urine receiving component. The entry-port connects to a tube that flows the urine from the urinary incontinence treatment device to collection vessel, typically a bag. Along the external wall of the tube are at least one, typically two or more, circumferential slits.

c) A vaginal stabilizing component that can have any spatial configuration that facilitates easy insertion of the component into the vagina and prevents its spontaneous expulsion from its placement-position within the vagina. Typically vaginal stabilization component has a spatial configuration of an elongated plate with the shape of, but is not limited to, a flat guitar, or, a flat rowing-paddle, or flat tennis racket. In addition, the vaginal stabilizing component can have any spatial configuration that facilities a tight yet comfortable connection of the component to the wall of a treated patient. The term “tight” in the context of the vaginal stabilizing component refers to reversibly fastened-connection the vaginal stabilizing component to the wall of the vagina, so as to allow little free motion of the urine collecting component. Comfortable in the context of the vaginal stabilizing component refers to not causing any physically unpleasant feelings to the treated patient.

The vaginal stabilizing component is connected to the rim of urine-receiving component on the far side from air-inlet hole. The vaginal stabilizing component can optionally be connected directly to the tube and to urine-receiving component.

The vaginal stabilizing component and the urine-receiving component of the urine collecting component are connected in an approximate perpendicular connection, in a configuration that positions the rim of urine-receiving component over the orifice of the uretheral tract when the vaginal stabilizing component is pressed towards the upwards directional wall of a treated patient.

The stabilizing component comprises (designated below as d to f):

d) A urine-receiving-component-support-ring that is constructed of a flat-ring which has inner spoke-bars that support at the center of the ring a thin plate-structure that has in its center at least one hole. Slits that extend from the rim of the hole provide the rim of the hole elasticity that enables the penetration of a tube having a diameter that fits into hole while slightly bending the rims of the hole. The ring-receiving-component-support-ring, can optionally have an elliptical configuration.

e) A connection-ring that has an adhesive layer on both its flat surface-sides.

f) A body-connection-pad that typically has an elongated configuration and has an adhesive sticky side and a plain (not sticky) side. Optionally, the sticky side reversibly connects to a body-connection-pad protective cover that maintains the stickiness of the body-connection pad prior to the deployment of the device of the present invention.

The urine-receiving-component-support-ring is connected by the connection-ring to the none sticky side of the body-connection-pad).

The urine collecting component and the stabilizing component of the external urinary incontinence treatment device of the present invention are reversibly connected together by inserting the tube of the urine collecting component into the hole in the plate-structure in the stabilizing component. The tube slides in the hole till the rim of the hole interlocks with one of the slits in the external circumference of the tube, thus, is fixated in place inside the hole. By squeezing simultaneously both sides of the plate structure, the tube is released from the interlocking and can be moved in the hole to interlock with another slit.

In deploying the external urinary incontinence treatment device of the present invention the urine collecting component and the stabilizing component are connected in a configuration that tube is fixated in place that is assumed to be the appropriate for the urinary incontinence treatment. If the position of the tube is found to be inappropriate (meaning the distance between the two components is inappropriate, causing an uncomfortable feeling)) it can easily be changed in the course of the treatment. The distance between the urine collecting component and the stabilizing component determines the extent of penetration of the vaginal stabilizing component into the vagina and the pressure applied to the body-connection-pad. When the two components are connected the vaginal stabilizing component is inserted into the vagina. The flat rowing-paddle or flat tennis racket configuration improves the anchoring of the vaginal stabilizing component in the vagina. The vaginal stabilizing component is pressed towards the wall of the vagina and the symphysis pubis behind the wall of the vagina. The pressing tightly fastens the reversible connection of the vaginal stabilizing component to the vagina wall. The placing of the vaginal stabilizing component in the vagina positions the connected urine receiving component over the orifice of the uretheral tract outside of the vagina. The simplicity of positioning of the receiving component over the orifice prevents the guessing if the urine receiving component is in its proper location. By pressing the urine receiving component towards the skin surrounding the orifice towards the body of the treated patient (done by pushing the tube of the urine collecting component) a urine leak free connection is established. With the urine collecting component inside the body of the patient, the body-connection-pad protective cover is removed and the body-connection-pad is connected to the skin in the genital area surrounding the external urinary incontinence treatment device. The connection to the skin fixates and stabilizes the device, thus, a tube for urine removal of urine is connected to the tube of the urine collecting component.

The connection of the body-connection-pad to the genital area of the treated patient anchors the stabilizing component of urinary incontinence treatment device of the present invention to body of the treated patient. When the tube of the urine collecting component is pressed towards the orifice of the uretheral tract the pressure applied to the skin surrounding the orifice (by the connected urine receiving component) is also applied to the urine-receiving-component-support-ring of the stabilizing component. The anchoring of the stabilizing component creates a fixed in place, stable platform, that facilities the transmission of the pressure from the urine-receiving-component-support-ring to the body-connection-pad that distributes the pressure to the connection between the pad and the skin of the patient.

When deploying the external urinary incontinence treatment device of the present invention, the stream of urine in the tube flowing the urine from the device towards a collection vessel creates a vacuum in the urine receiving component. When no urine enters the urine receiving component after the tube removing the urine was at least partially filled, the vacuum created by the urine that remains in the tube does not enable the continued streaming of the urine towards the collection vessel. The optional air-inlet hole enables ambient air to enter the urine receiving component, thus, eliminating the vacuum and enabling the continual removal of the urine by the urine removal tube. The air-inlet hole is designed so as to enable the passage of air and eliminated the passage of water (urine) molecules.

The method the treatment of urinary incontinence treatment women of the present invention is deploying an external urinary incontinence treatment device that minimized the discomfort associated with the connection of a device to the body and simplifies the location of the orifice of urethral tract of the treated patient for the connection of a urine receiving component around the orifice.

The deployed device is constructed of two components: a stabilizing component and urine collecting component.

The stabilizing component comprises: a urine-receiving-component-support-ring, a connection-ring and a body-connection-pad.

The connection ring is a flat configured ring that has an adhesive layer on both its surface-sides. The body connection pad comprises an elongated sheet of flexible material with a layer of an adhesive material in one of its sides and a hole at its center. The urine-receiving-component-support-ring has a ring configuration and tube support plate in its center. The tube support plate connects to the rim of said urine-receiving-component-support-ring by at least two spokes. The tube support plate has at in it at least one hole with slits running from the rim of the hole. The connection-ring of the stabilizing component connects to urine-receiving-component-support-ring and to the body-connection-pad.

The urine collecting component comprises: a urine receiving component, a tube and a vaginal stabilizing component.

The tube has least one circumferential slit along its external wall and an entry port at its tip. The vaginal stabilizing component comprises a plate having an elongated configuration, The vaginal stabilizing component and the tube are connected to the a urine receiving component and the tube communicates freely with the urine receiving component. The tube inserts into said hole in the tube support plate in said urine-receiving-component-support-ring, the tube moves freely inside said hole until the slit in in tube reversibly interlocks with the rim of the hole in the tube support plate.

In deploying the device of the present invention, the vaginal stabilizing component is inserted into vagina of said treated patient and the rim of said urine receiving component reversibly tightly connects to the skin surrounding the urethral orifice of said treated patient while the body-connection-pad reversibly connects to the skin in the genital region of the treated patient.

The distance between the stabilizing component and the urine collecting component and be easily and reversibly changed and adjusted to a configuration that will be most comfortable to the treated patient. The distance between the components determines the pressure applied to the body-connection-pad and the depth of insertion of the vaginal stabilizing component into the vagina of a treated patient.

The spatial configuration of the urine collecting component determines that when the vaginal stabilizing component is tightly connected to the wall of the vagina the urine receiving component is just above the orifice of the urethral tract. By pressing the vaginal stabilizing component into the body of the treated patient the rim of the urine receiving component connect with the skin surrounding the orifice. Thus, method of the present invention prevents the guessing of where the to exactly position the urine receiving component of an external urinary incontinence treatment in the treatment of women.

### BRIEF DESCRIPTION OF THE DRAWINGS

In order to better understand the present invention, and appreciate its practical applications, the following Figures are provided and referenced hereafter. It should be noted that the Figures are given as examples only and in no way limit the scope of the invention. Like components are denoted by like reference numerals.

FIG. 1 is an isometric from-above-and-side view of an illustration of an external urinary incontinence treatment device of the present invention, with the components of the device disassembled and disconnected.

FIG. 2 is an isometric from-above-and-side view of an illustration of an external urinary incontinence treatment device of the present invention in an assembled and connected configuration.

FIG. 3 is an isometric from-above-and-side view of an illustration of a urine-receiving-component-support-ring (URCSR) and a urine-collecting component (UCC) of the present invention, in a disconnected configuration, without the body connection pad, and the connection ring.

FIG. 4 is an isometric from-above-and-side view of an illustration of the URCSR and the UCC shown in FIG. 3, in a connected configuration.

FIG. 5A up to and including FIG. 5F, are illustrations of consecutive stages of deploying the external urinary incontinence treatment device of the present invention, illustrated in FIG. 2.

FIG. 6 is a cross cut illustration of the external urinary incontinence treatment device of the present invention deployed in a treated patient.

### DETAILED DESCRIPTION OF A PREFERRED EMBODIMENT

It should be clear that the description of the embodiment and attached Figures set forth in this specification serves only for a better understanding of the invention, without limiting its scope.

The external urinary incontinence treatment device of the present invention (10) is composed of: a stabilizing component (12) referred to interchangeably as SC, and a urine collecting component (26), referred to interchangeably as UCC.

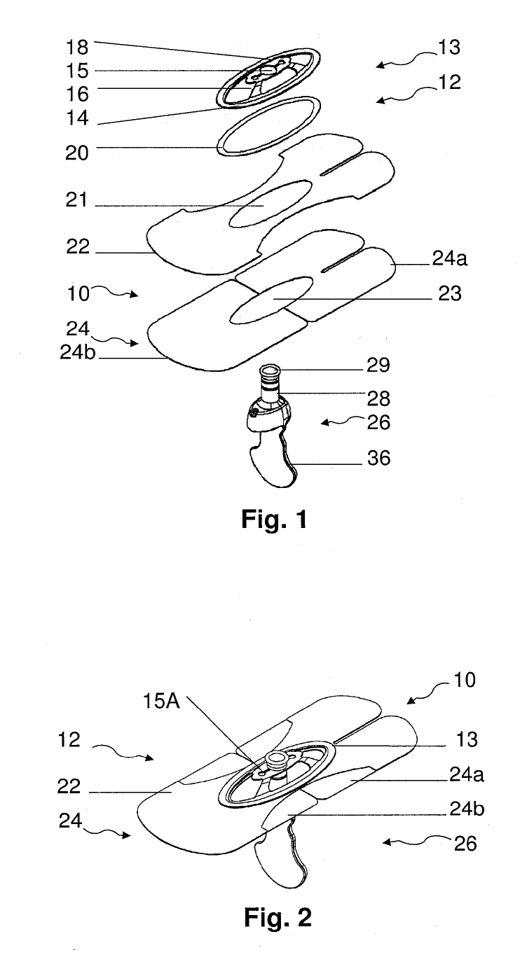

FIG. 1 shows an isometric from-above-and-side view illustration of an external urinary incontinence treatment device (10) of the present invention, with the components that construct SC (12) disassembled and in a configuration in which SC (12) is apart from the UCC (26), and the SC (12) is illustrated in disassembled configuration, illustrating connection pad (22) and connection ring (20).

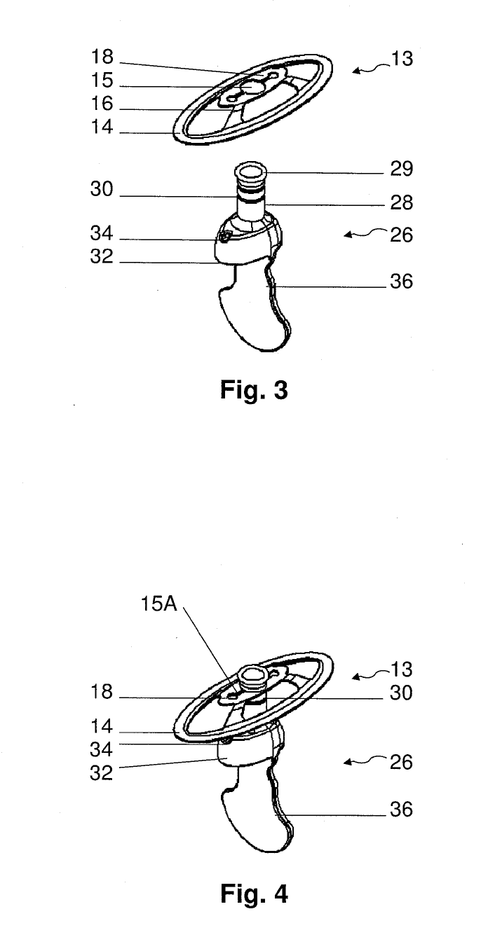

FIG. 2 shows an isometric from-above-and-side view illustration of the external urinary incontinence treatment device illustrated in FIG. 1, in a configuration in which the SC (12) components are assembled and the SC (12) reversibly connected to the UCC (26).

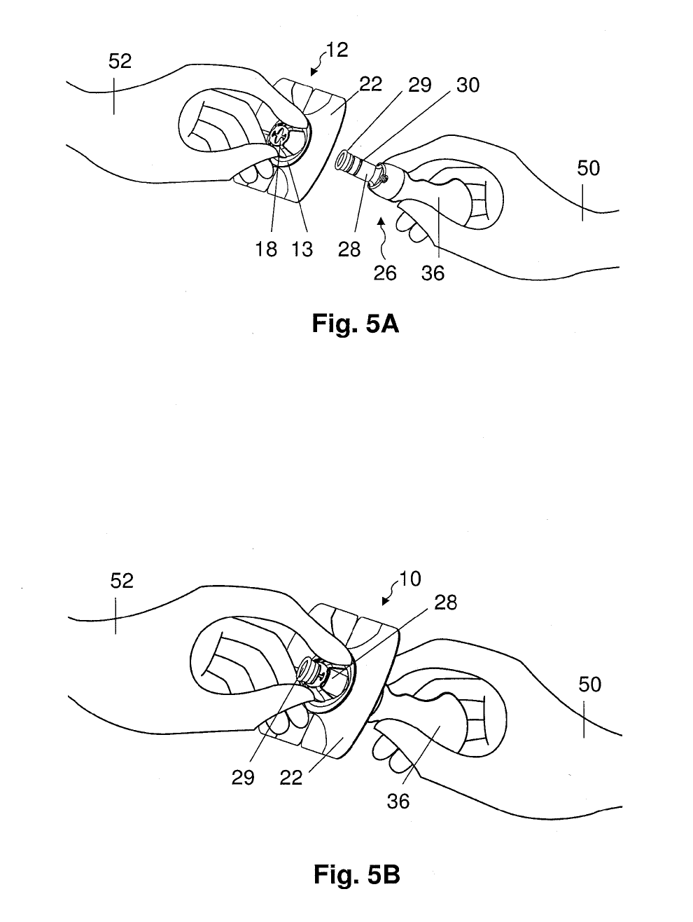

FIG. 3 is an isometric from-above-and-side view of an illustration of a urine-receiving-component-support-ring, referred to interchangeably as URCSR (13), which is a component of SC (12), and a urine-collecting component (UCC) (26), illustrated in FIG. 1.

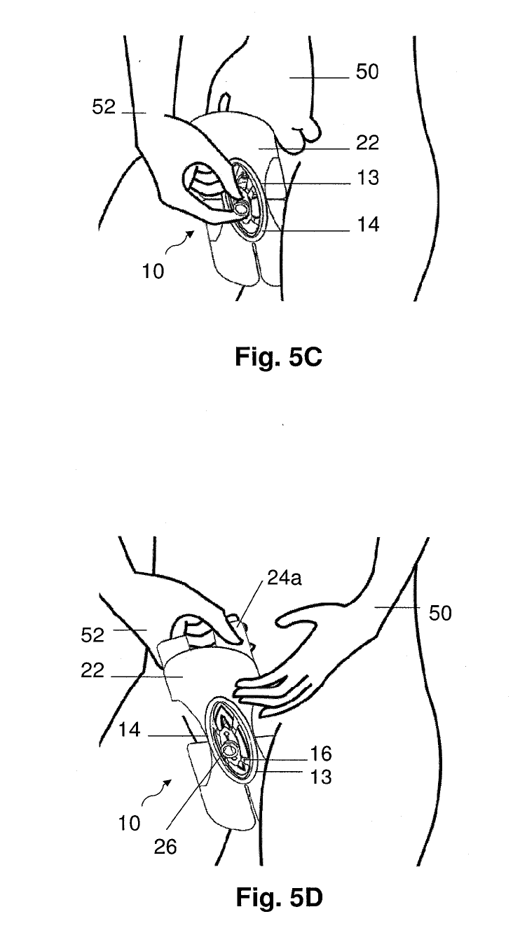

FIG. 4 is an isometric from-above-and-side view of an illustration of the URCSR (13) and UCC (26), shown in FIG. 1, in a reversible connected configuration.

Reference is presently made to stabilizing component (12) of the present invention shown in a disassembled configuration in FIG. 1 and in an assembled configuration in FIG. 2.

SC (12) is constructed of a URCSR (13), a connection-ring (20), a body-connection-pad, referred to interchangeably as BCP (22) and a BCP protective cover, referred to interchangeably as BCPPC (24). URCSR (13) is constructed of a flat-ring (14) which has inner spoke-bars (16) that support at the center, or near the center, of the ring a plate-structure (18) that has in its center at list one hole (15). URCRS (13), optionally has a flat elliptical configuration, In the illustrate embodiment 4 spoke-bars (16) are illustrated. At least two slits (15A) extend from the rims of hole (15). URCSR (13) is constructed of a rigid or semi-rigid material, such as, but not limited to, silicon, plastic or rubber. The slits (15A) extending from hole (15) in plate (18) provide the rim of the hole elasticity that enables the penetration of a tube having a diameter that fits into hole (15) while slightly bending the rim of the hole. The bent rim of the hole interlock into circumference slits (30) along tube (28). By squeezing the longitudinal sides of plate structure (18) towards each other with tube (28) interlocked, the configurational change along slits (15A) releases tube (28) from the interlocked state, and tube (28) can (again) moved inside hole (15). If plate (18) is not squeezed, tube ((28) can be moved till another slit (30) interlocks with the tube. Connection-ring (20) is a flat ring constructed of a rigid or semi-rigid material, such as, but not limited to, silicon, plastic or rubber, or thin sheet of glue. Connection ring (20) has an adhesive layer on both its surface-sides. The adhesive layers are composed of a thin spread of sticky glue material, or, alternatively, composed of sheets made of, but not limited to, thin-paper or thin plastic material, that are connected to the surface-sides and have a layer of adhesive material. Connection ring (20) connects to ring (14) in one side and around hole (21) in the top, none sticky side, of body connection pad (22). BCP (22) is an elongated pad made of a sheet of flexible material such as, but not limited to, cloth or plastic material or paper, and has a hole (21), typically at its center, or near the center. Typically, hole (21) has an elliptical configuration. The side of BCP (22) that connects to connection-ring (20) is plain (not sticky) and the other side is coated by a human-skin contact-suitable adhesive sticky layer, that connects BCP (22) firmly yet reversibly, to a treated patient. The sticky layer can be formed by a glue layer on the surface of the pad, or alternatively, by a thin film with a fixated side connected to the BCP and a sticky side left free. BCP protective cover (24) is reversible connected to the sticky side of BCP (22) and BCPPC (24) is removed when the device (10) of the present invention is deployed. BCPPC (24) is divided into two parts, (24A) and (24B), which encircle a hole (23) that aligns with hole (21) when the two parts are connected to BCP (22). The partition to two parts enables the peeling off and removal of BCPPC (24) from BCP (22) without the necessity of changing the placement of BCP (22) when device (10) is deployed. The rim of hole (23) is reversibly connected to the rim of hole (21).

Reference is presently made to the urine-collecting component (26), illustrated in detail in FIG. 1 and FIG. 3.

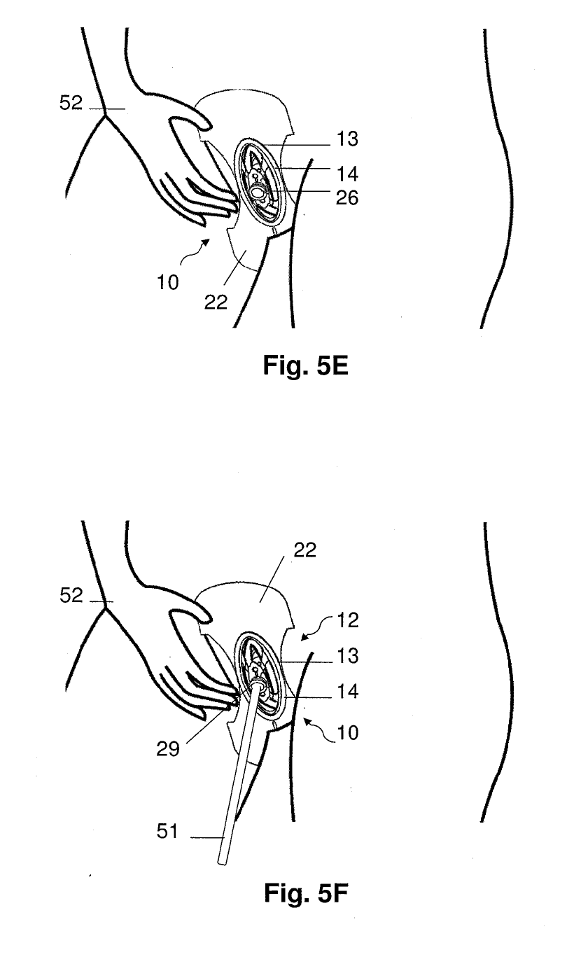

UCC (26) is typically produced as, but not limited to, a component having a single entity made of rigid and semi-rigid material or materials such as, but not limited to, silicon and/or plastic and/or rubber. UCC is constructed of a urine-receiving component (32), referred interchangeably as URC, that has a tube-entry hole at one of its sides and an air-inlet hole (34) at its opposite side. Air-inlet-hole (34) is a hole that runs through the wall of urine-receiving component (32) and has a diameter of approximate between 0.3 and 0.6 millimeter. The diameter of hole (34) enables the free passage of air and restricts the passage of urine. A tube (28) connects to the tube entry hole in URC (32) and communicates freely with the enclosed volume of the urine receiving component (32). Tube (28) has at the tip not connected to the URC (32), a rigid, or semi rigid, ring tube entry port (29) for the connection to a urine removal tube (51) (shown in FIG. 5F) that flows the urine to a collection vessel. In addition, tube (28) has along its external wall at least one, typically two or more, circumference slits (30). A vaginal stabilizing component (36), referred to interchangeably as VSC, is connected to the rim of urine-receiving component (32) on the far side from air-inlet hole (34). VSC (36) can have any spatial configuration that facilitates easy insertion of the component into the vagina and prevents its spontaneous expulsion from its placement-position within the vagina. Typically VSC (36) has a spatial configuration of an elongated plate with the shape of, but is not limited to, a flat guitar, or, a flat rowing-paddle, or flat tennis racket. Optionally, air-inlet hole is not limited to the far side of component (32) and can be in other locations in the component or in the tube (28).

Typically, but not limited to, the urine receiving component is made in an elongated dome configuration that fits the spatial configuration of the genital region where the receiving component is deployed. In describing the embodiment of the present invention illustrated in the figures the term “urine-receiving component” is interchangeably used in the text as the “urine-receiving dome”.

Reference is presently made to explaining the reversible connection of URCSR (13) of SC (12) to tube (28) of UCC (26), illustrated in FIG. 3 and FIG. 4. Tube (28) is inserted into hole (15 in plate structure (18) of urine-receiving-component-support-ring (13). The tube is slid in the hole and is reversibly fixated in place by the rim of hole (15), interlocking with the slit (30) of choice along the length of the tube (28). The choice of the slit is determined by the patient and can be altered in the course of the treatment.

Reference is presently made to explaining the procedural stages of deploying the urinary incontinence treatment device (10) shown in FIG. 2, illustrated in FIG. 5A up to and including FIG. 5F.

FIG. 5A illustrates a stabilizing component (12) held by one hand (52) of a patient and a urine collecting component (26), held by the other hand (50). Hole (18) in SC (12) is aligned with entry port (29) of tube (28) in UCC (26).

FIG. 5B illustrates the insertion of tube (28) of UCC (26) illustrated in 5A, into hole (15) in URCSR (13). Slit (30), shown in FIG. 5A, interlocks reversibly with the rim of hole (15) in URCSR (13). The choice of the slit (30) used is made by the patient (or care giver) and can be easily changed if another slit is more suitable for fixating the movement of tube (30) inside hole (15). The length of tube (28) extending from the URC (32) towards the URCSR (13) determines the fastness of the connection between the urine receiving component (32) and the surrounding skin of the orifice of the uretheral tract of the treated patient. The fastness is applied to the extent that causes minimum discomfort to the treated patient.

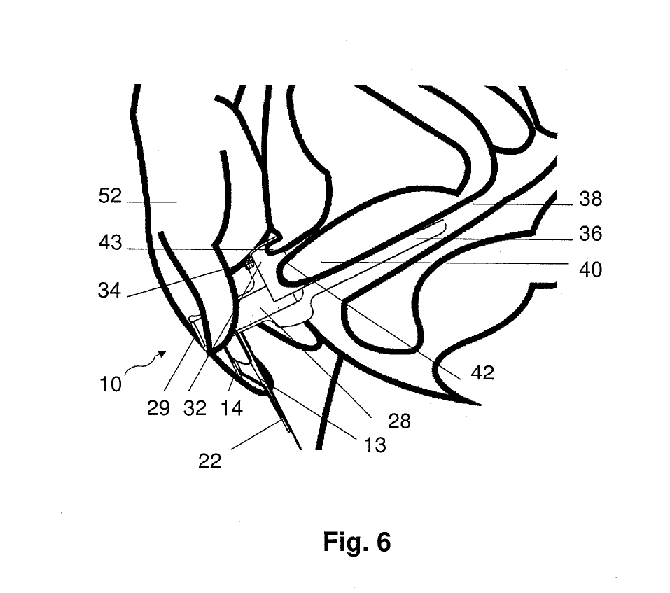

FIG. 5C illustrates the patient using her hand (50) to part the lips of the labia minora (not shown in the Fig.) and inserting vaginal stabilizing component (36) of the UCC (26), into the vagina ((38), (shown in FIG. 6)). The insertion is done by holding tube (28) with her other hand (52) and pushing the VSC (36) into the vagina. Ring (14) of SC (12) is shown connected to body connection pad (22). By pushing the VSC (36) towards the wall of the vagina and towards the symphysis pubis (40), as illustrated in FIG. 6, the rim of urine receiving dome (32) is positioned over and around the orifice of the uretheral tract of the patient. By continuing to push UCC (26) urine receiving dome is fastened to the skin surrounding the orifice. By movements of the UCC (26) toward the body, while the VSC (36) is in the vagina, the connection, adjustment and fastening of the urine receiving dome (32) are achieved. The fixating of the urine receiving dome (32) around the orifice of the uretheral tract is achieved by connecting BCP to the body of the treated patient, as illustrated in FIG. 5D and FIG. 5E.

FIG. 5D illustrates the patient peeling BCP protective cover (24) from body connection pad (22) while vaginal stabilizing component (36) is in place in her vagina, and the URC (32) is surrounding the urethra hall. The patient is shown pressing BCP (22) towards her body with one hand (50) while pulling and peeling BCPPC (24) from BCP (22) with the other hand (52).

FIG. 5E illustrates the patient pressing with her hand (52) BCP (22), towards her body. With BCPPC (24) removed, the adhesive layer on BCP (22) reversibly connects to the skin of the patient, thus, stabilizing device (10) in its placement from outside the body. The inserted vagina stabilizing component (36) stabilizes device (10) in its placement from inside the body of the patient.

By squeezing simultaneously towards each other the longitudinal sides of plate-structure (18) (shown in FIG. 1) tube (28) can be freed from being interlocked in a given position with the rim of hole (15) and another interlocked position along the length of tube (28) can be easily established by moving tube (28) inside hole (15) either towards or from the body of the treated patient.

FIG. 5F illustrates device (10) stabilized in the body of the patient and a urine removal tube (51) connected to tube entry port (29) of tube (28).

By releasing the interlock slit (30) of tube (28) from the rim of hole (15) in plate structure (18) the tight connection between the urine receiving dome (32) and the surrounding of the orifice of the urethral tract (43), (shown in FIG. 6) is terminated. By disconnecting the BCP from the body and slightly swirling the VSC (36) inside the vagina the UCC can be withdrawn from the body of the treated patient and device 10 completely removed.

FIG. 6 is a cross cut illustration of the external urinary incontinence treatment device (10) of the present invention deployed in a treated patient. The illustration in FIG. 5E is the view from outside of the body of the patient of the deployment-state of device (10) shown in FIG. 6. The illustration shows plate of VSC (36) of the UCC (26) inside the vagina (38) of a treated patient and pressed the wall of the vagina towards the symphysis pubis (40). With plate (36) pressed towards the symphysis pubis (40), urine-receiving dome (32) is pressed towards the body of the patient by pressing on tube (28), thus forming a reversible urine leak-free connection between the rim of urine-receiving dome (32) and the skin surrounding the external ureteral orifice (43) of the treated patient. Plate (36) pressed inside the vagina together with body-connection-pad (22) reversibly connected to the external skin surrounding the genital region of the treated patient secure the reversible fixating of the urine receiving dome (32) in place.

It should also be clear that a person skilled in the art, after reading the present specification could make adjustments or amendments to the attached Figures and above described embodiments that would still be covered by the present invention

I claim:

1) An external urinary incontinence treatment device for a woman patient comprising:
a stabilizing component,
a urine collecting component,
wherein said stabilizing component comprises:
a urine-receiving-component-support-ring,
a connection-ring,
a body-connection-pad,
wherein, said connection ring has an adhesive layer on both its surface-sides,
wherein, said body connection pad comprises an elongated sheet of flexible material with a layer of an adhesive material in one of its sides and a hole at its center,
wherein, said urine-receiving-component-support-ring has a ring configuration and tube support plate in its center,
wherein said tube support plate connects to the rim of said urine-receiving-component-support-ring by at least two spokes,
wherein said tube support plate has at in it at least one hole with slits running from the rim of the hole,
wherein, said connection-ring of said stabilizing component connects to urine-receiving-component-support-ring and to said body-connection-pad,
wherein said urine collecting component comprises:
a urine receiving component,
a tube,
a vaginal stabilizing component,
wherein, said tube has least one circumferential slit along its external wall and an entry port at its tip.
wherein, said vaginal stabilizing component comprises a plate having an elongated configuration,
wherein, said vaginal stabilizing component and said tube are connected to said a urine receiving component and said tube communicates freely with said urine receiving component,
wherein, said tube inserts into said hole in said tube support plate in said urine-receiving-component-support-ring, said tube moves freely inside said hole until said slit in said tube reversibly interlocks with the rim of said hole in said tube support plate,
wherein, said vaginal stabilizing component is inserted into vagina of said treated patient and the rim of said urine receiving component reversibly tightly connects to the skin surrounding the urethral orifice of said treated patient,
wherein, said body-connection-pad reversibly connects to the skin in the genital region of said treated patient.

2) The stabilizing component of external urinary incontinence treatment device of claim 1, wherein the stabilizing component is produced as a single entity component.

3) The urine collecting component, of external urinary incontinence treatment device of claim 1, wherein the urine collecting component is produced as a single entity component.

4) The stabilizing urine-receiving-component-support-ring of stabilizing component of external urinary incontinence treatment device of claim 1 wherein the stabilizing urine-receiving-component-support-ring is made of semi-rigid materials.

5) The body-connection-pad of stabilizing component of external urinary incontinence treatment device of claim 1 wherein the body-connection-pad is made of a material having a flexible sheet configuration.

6) The body-connection-pad of stabilizing component of external urinary incontinence treatment device of claim 1 wherein the body-connection-pad is reversibly connected to a body-connection-pad protective cover.

7) The urine collecting component of external urinary incontinence treatment device of claim 1 wherein the urine collecting component is made of semi-rigid materials.

8) The urine receiving component of urine collecting component of external urinary incontinence treatment device of claim 1, wherein the urine receiving component has a spatial configuration that facilitates a reversible urine leak-tight connection with the skin surrounding the orifice of a treated patient.

9) The vaginal stabilizing component of the urine collecting component of external urinary incontinence treatment device of claim 1 wherein the vaginal stabilizing component has an elongated plate spatial configuration.

10) The vaginal stabilizing component of claim 9 wherein the vaginal stabilizing component has a spatial configuration that prevents the spontaneous expulsion from within the vagina of a treated patient.

11) The vaginal stabilizing component of claim 9 wherein the vaginal stabilizing component has a spatial configuration that facilitates a tight reversible yet comfortable connection with the wall of the vagina of a treated patient.

12) The urine receiving component of urine collecting component of external urinary incontinence treatment device of claim 1, wherein the urine receiving component has an air inlet hole running through the wall of the urine receiving component.

13) The tube of urine collecting component of external urinary incontinence treatment device of claim 1, wherein the tube has an air inlet hole running through the wall of the tube.

14) A method for the treatment of urinary incontinence treatment of a women by deploying a external urinary incontinence treatment device,
the device comprising: a stabilizing component and urine collecting component, wherein said stabilizing component comprises:
a urine-receiving-component-support-ring,
a connection-ring,
a body-connection-pad,
wherein, said connection ring has an adhesive layer on both its surface-sides,
wherein, said body connection pad comprises an elongated sheet of flexible material with a layer of an adhesive material in one of its sides and a hole at its center,
wherein, said urine-receiving-component-support-ring has a ring configuration and tube support plate in its center,
wherein said tube support plate connects to the rim of said urine-receiving-component-support-ring by at least two spokes,
wherein said tube support plate has at in it at least one hole with slits running from the rim of the hole,
wherein, said connection-ring of said stabilizing component connects to urine-receiving-component-support-ring and to said body-connection-pad,
wherein said urine collecting component comprises:
a urine receiving component,
a tube,
a vaginal stabilizing component,
wherein, said tube has least one circumferential slit along its external wall and an entry port at its tip.
wherein, said vaginal stabilizing component comprises a plate having an elongated configuration,
wherein, said vaginal stabilizing component and said tube are connected to said a urine receiving component and said tube communicates freely with said urine receiving component,
wherein, said tube inserts into said hole in said tube support plate in said urine-receiving-component-support-ring, said tube moves freely inside said hole until said slit in said tube reversibly interlocks with the rim of said hole in said tube support plate,
wherein, said vaginal stabilizing component is inserted into vagina of said treated patient and the rim of said urine receiving component reversibly tightly connects to the skin surrounding the urethral orifice of said treated patient,
wherein, said body-connection-pad reversibly connects to the skin in the genital region of said treated patient.
